(19) United States
(12) Patent Application Publication
(10) Pub. No.: US 2025/0137091 A1
(43) Pub. Date: May 1, 2025

(54) PROCESSES FOR RECOVERING RHODIUM FROM HYDROFORMYLATION PROCESSES

(71) Applicant: Dow Technology Investments LLC, Midland, MI (US)

(72) Inventors: Michael A. Brammer, Lake Jackson, TX (US); Yujun Liu, Pearland, TX (US); Amarnath Singh, Pearland, TX (US); Glenn A. Miller, South Charleston, WV (US); Adrian Francis Backes, London (GB); Michael Betham, Stockton-on-Tees (GB)

(21) Appl. No.: 18/686,705

(22) PCT Filed: Oct. 13, 2022
(86) PCT No.: PCT/US2022/078046

§ 371 Date: Feb. 26, 2024

(51) Int. Cl. C22B 11/00; C07C 45/78; C22B 1/02; C22B 7/00

(52) U.S. Cl. CPC C22B 11/048; C07C 45/786; C22B 1/02; C22B 7/005

(57) ABSTRACT

The process disclosure relates to processes for recovering rhodium from a hydroformylation process. In one aspect, a tails stream from a product-catalyst separation zone is provided to at least one organic solvent nanofiltration separation membrane, wherein a final permeate stream exits a final organic solvent nanofiltration separation membrane, wherein the rhodium concentration in the final permeate stream is lower than the rhodium concentration in the tails stream. The permeate stream is incinerated on-site to create a rhodium-containing ash.

### FIELD

The present invention relates generally to processes for recovering rhodium from hydroformylation processes.

### INTRODUCTION

Higher alcohols (carbon chain length of 7 or longer) may be manufactured via hydroformylation of higher olefins using a homogeneous transition metal catalyst.

Employing a catalyst comprised of rhodium in such processes allows efficient operation at relatively low temperatures and pressures. In a typical process, the reaction fluid containing aldehyde intermediates and the homogeneous catalyst is fed to a separation zone wherein the crude product aldehydes are vaporized and condensed overhead, and the non-volatile effluent (which contains the catalyst) is recycled to the reaction zone. During continuous operation of these processes, the aldehyde compounds will typically form heavy byproducts (often referred to as heavy ends or heavies) due to aldol condensation reactions. The low volatility of these compounds precludes their removal via vaporization; thus, the heavy byproducts or heavies will accumulate over time in the reaction zone. Controlling the heavies concentration in the system requires that the heavies be removed via liquid purge (e.g., removing a portion of the non-volatile effluent from the separation zone). This fluid will of course contain the homogeneous rhodium catalyst, which is quite expensive.

As discussed in PCT Publication No. WO2020/263462, the purged fluid may be unstable and degrade upon storage. The fluid must be collected in a shipping container and is often stored for a lengthy period prior to shipping to a precious metal recovery facility. During this time, the catalyst may precipitate from the solution which greatly complicates the precious metal recovery process. Furthermore, precious metal being stored in a non-productive form (i.e., not directly being used for aldehyde manufacture) is an expense which must be accounted for. In addition, many regions consider “spent catalyst fluid” to be a hazardous waste, which can complicate shipping.

Given the high price of rhodium, it would be desirable to have a cost-efficient processes to control the heavies concentration in higher olefins hydroformylation processes.

### SUMMARY

The present invention provides processes for recovering rhodium from hydroformylation processes. In some aspects, the processes are particularly advantageous in improving rhodium accountability while also maintaining the heavies concentration within a higher olefins hydroformylation process.

In one embodiment, a process for recovering rhodium from a hydroformylation process that comprises producing at least one aldehyde in a reaction zone, the reaction zone comprising a C₆ to C₂₂ olefin, hydrogen and carbon monoxide in the presence of a catalyst, wherein the catalyst comprises rhodium and an organophosphorus ligand, comprises:
(a) receiving a tails stream from a product-catalyst separation zone, wherein the tails stream comprises aldehydes, heavies, rhodium, and an organophosphorus ligand;
(b) providing at least a portion of the tails stream to at least one organic solvent nanofiltration (“OSN”) separation membrane, wherein a final permeate stream exits a final OSN separation membrane, the final permeate stream comprising aldehydes, heavies, rhodium, and an organophosphorus ligand, wherein the rhodium concentration in the final permeate stream is lower than the rhodium concentration in the tails stream; and
(c) incinerating the final permeate stream on-site to create a rhodium-containing ash.

These and other embodiments are described in more detail in the Detailed Description.

### BRIEF DESCRIPTION OF THE FIGURES

FIG. 1 is a schematic of a system for implementing some embodiments of processes of the present invention.

FIG. 2 is a schematic of a lab scale apparatus used for the experiments described in the Examples section.

### DETAILED DESCRIPTION

The disclosed process is used in conjunction with a hydroformylation process that comprises contacting CO, H₂, and a C₆ to C₂₂ olefin under hydroformylation conditions sufficient to form at least one aldehyde product in the presence of a catalyst comprising, as components, rhodium and an organophosphorous ligand.

All references to the Periodic Table of the Elements and the various groups therein are to the version published in the CRC Handbook of Chemistry and Physics, 72nd Ed. (1991-1992) CRC Press, at page I-11.

Unless stated to the contrary, or implicit from the context, all parts and percentages are based on weight and all test methods are current as of the filing date of this application. For purposes of United States patent practice, the contents of any referenced patent, patent application or publication are incorporated by reference in their entirety (or its equivalent US version is so incorporated by reference) especially with respect to the disclosure of definitions (to the extent not inconsistent with any definitions specifically provided in this disclosure) and general knowledge in the art.

As used herein, “a,” “an,” “the,” “at least one,” and “one or more” are used interchangeably. The terms “comprises,” “includes,” and variations thereof do not have a limiting meaning where these terms appear in the description and claims.

Also herein, the recitations of numerical ranges by endpoints include all numbers subsumed in that range (e.g., 1 to 5 includes 1, 1.5, 2, 2.75, 3, 3.80, 4, 5, etc.). For the purposes of the invention, it is to be understood, consistent with what one of ordinary skill in the art would understand, that a numerical range is intended to include and support all possible subranges that are included in that range. For example, the range from 1 to 100 is intended to convey from 1.01 to 100, from 1 to 99.99, from 1.01 to 99.99, from 40 to 60, from 1 to 55, etc.

As used herein, the term “ppmw” means parts per million by weight.

For purposes of this invention, the term “hydrocarbon” is contemplated to include all permissible compounds having at least one hydrogen and one carbon atom. Such permissible compounds may also have one or more heteroatoms. In a broad aspect, the permissible hydrocarbons include acyclic (with or without heteroatoms) and cyclic, branched and unbranched, carbocyclic and heterocyclic, aromatic and nonaromatic organic compounds that can be substituted or unsubstituted.

As used herein, the term “substituted” is contemplated to include all permissible substituents of organic compounds unless otherwise indicated. In a broad aspect, the permissible substituents include acyclic and cyclic, branched and unbranched, carbocyclic and heterocyclic, aromatic and nonaromatic substituents of organic compounds. Illustrative substituents include, for example, alkyl, alkyloxy, aryl, aryloxy, hydroxyalkyl, aminoalkyl, in which the number of carbons can range from 1 to 20 or more, preferably from 1 to 12, as well as hydroxy, halo, and amino. The permissible substituents can be one or more and the same or different for appropriate organic compounds. This invention is not intended to be limited in any manner by the permissible substituents of organic compounds.

As used herein, the term “hydroformylation” is contemplated to include the conversion of at least one olefin to a mixture of aldehydes using synthesis gas in the presence of one or more rhodium complex catalysts.

For the purposes of this invention, the terms “reaction zone” and “reactor” are used interchangeably and refer to a region of the hydroformylation process containing the reaction fluid and wherein both olefins and synthesis gas are added at elevated temperatures.

For the purposes of this invention, the terms “product-catalyst separation zone” and “separation zone” are used interchangeably and refer to a region where the reaction fluid is separated into (1) a crude aldehyde product stream which is predominantly free of the rhodium catalyst and is provided to further downstream operations such as hydrogenation, aldolization, etc., and (2) a tails stream which contains the rhodium catalyst. In one embodiment, the product-catalyst separation zone comprises a vaporizer, wherein the reaction fluid is heated (i.e., the temperature is higher than the reaction zone temperature) causing an increase in the vapor pressure of the product aldehyde. The product-catalyst separation zone may optionally be operated at reduced pressure. In one embodiment, the vaporizer features flowing gas of varying composition that aids in product removal and optionally helps stabilize the catalyst (“strip gas vaporizer”). The resulting gaseous phase is then passed through a condenser to provide a liquid crude aldehyde product stream and a non-volatile effluent tails stream (tails, vaporizer tails, or tails stream) which contains the rhodium complex catalyst. Examples of such strip gas vaporizers are described, for example, in U.S. Pat. Nos. 8,404,903 and 10,023,516. In one embodiment, the product-catalyst separation zone comprises at least one organic solvent nanofiltration stage. In one embodiment, the product-catalyst separation zone comprises at least one organic solvent nanofiltration stage in combination with a vaporizer. In addition to a strip gas vaporizer, other examples of product-catalyst separation zone can include solvent extraction, crystallization, distillation, wiped film evaporation, falling film evaporation, phase separation, filtration, or any combination thereof.

As used herein, the term “tails stream” is contemplated to include the rhodium catalyst-containing effluent from the product-catalyst separation zone. In one embodiment where the product-catalyst separation zone comprises a strip gas vaporizer, the tails stream comprises the non-volatile effluent from the strip gas vaporizer.

As used herein, the term “OSN separation membrane” means an organic solvent nanofiltration separation membrane that is compatible with a tails stream from a product-catalyst separation zone and is capable of retaining a majority of the rhodium, the rhodium-ligand complex catalyst and optionally free organophosphorous ligand while at the same time allowing a portion of the heavies to pass through as permeate. Such OSN separation membranes include a surface active layer that comprises, for example and without limitation, polyldimethylsiloxane (PDMS), polyimides, polyamides, polyetheretherketone (PEEK), polypropylene, and the like. In some embodiments, the OSN separation membrane may further be comprised of a support material to which one or more surface active layers have been applied.

As used herein, the term “OSN stage” is contemplated to include providing at least a portion of the tails stream to a process step utilizing an OSN separation membrane to provide a retentate and a permeate stream. A single OSN stage may be employed in some embodiments. In some embodiments, at least two OSN stages are used and are conducted in parallel. In some embodiments, at least two OSN stages are used and conducted in series.

The term “OSN feed” is contemplated to include that portion of the rhodium catalyst-rich tails stream which is supplied to an OSN stage for nanofiltration. In some embodiments, the tails stream is the OSN feed. The “OSN feed rate” is contemplated to describe the volume per unit time at which an OSN feed is provided to an OSN stage.

The term “OSN retentate” or “retentate” is contemplated to include the portion of an OSN feed which does not pass through an OSN separation membrane. In general, the amount of fluid which will pass through an OSN separation membrane will depend on a plurality of factors, including but not limited to the nature of the OSN separation membrane itself, the properties of the fluid (e.g., viscosity), the process temperature, and the pressure applied to the feed side of the membrane. In some embodiments, the retentate may be returned to the reaction zone. In other embodiments, the retentate may be recycled and combined with the tails stream to comprise the OSN feed and thereby be provided to the same OSN stage in a semi-continuous or loop fashion. In some embodiments, the retentate may be provided to a second OSN stage in parallel or in series.

The term “OSN permeate” or “permeate stream” is contemplated to include the portion of an OSN feed which passes through OSN separation membrane and comprises aldehyde product and heavies; importantly the permeate stream contains a lower concentration of rhodium relative to the OSN feed. In some embodiments, the permeate stream contains a lower concentration of organophosphorous ligand relative to the feed. In some embodiments, two or more OSN stages are conducted in series, wherein the permeate stream from the first stage comprises the OSN feed for the second stage. The term “final permeate stream” refers to the permeate stream that exits the final (or only) OSN separation membrane and is sent for incineration.

The terms “reaction fluid,” “reaction medium” and “catalyst solution” are used interchangeably herein, and may include, but are not limited to, a mixture comprising: (a) a rhodium-organophosphorous complex catalyst, (b) free organophosphorous ligand, (c) aldehyde products formed in the reaction, (d) unreacted reactants, (e) heavies, (f) a solvent for said rhodium complex catalysts and said free phosphine ligands, and, optionally (g) organophosphorous ligand decomposition products such as the corresponding oxide. The reaction fluid can encompass, but is not limited to, (a) a fluid in a reaction zone, (b) a fluid stream on its way to a separation zone, (c) a fluid in a separation zone, (d) a tails stream, (e) the OSN feed, (f) the OSN permeate, (g) the OSN retentate, (h) a fluid withdrawn from a reaction zone or separation zone, (i) a fluid in an external cooler, and (j) a fluid in a catalyst treatment zone (e.g., an extractor).

As used herein the terms “rhodium catalyst”, “rhodium complex”, “rhodium complex catalyst”, and “catalyst complex” are used interchangeably and are contemplated to comprise at least one rhodium atom with ligands bound or coordinated via electron interaction. Examples of such ligands include but are not limited to bisphosphites, triphenylphosphine, tetradentate phosphines, triorganophosphites, carbon monoxide, olefin, and hydrogen.

As used herein, the term “free” ligand is contemplated to comprise phosphorous-containing molecules that are not bound or coordinated to rhodium.

As used herein, the terms “heavy byproducts” and “heavies” are used interchangeably and refer to byproducts that have a normal boiling point that is at least 25° C. above the normal boiling point of the desired product of the hydroformylation process. Such materials are known to form inherently in hydroformylation processes under normal operation through one or more side reactions, including for example, by aldol condensation or ligand degradation.

The present invention generally relates to processes for recovering rhodium from a hydroformylation process that comprises producing at least one aldehyde in a reaction zone, the reaction zone comprising a C₆ to C₂₂ olefin, hydrogen and carbon monoxide in the presence of a catalyst, wherein the catalyst comprises rhodium and an organophosphorus ligand. In one aspect, the process comprises:
(a) receiving a tails stream from a product-catalyst separation zone, wherein the tails stream comprises aldehydes, heavies, organophosphorous ligand, and rhodium;
(b) providing at least a portion of the tails stream to at least one organic solvent nanofiltration (OSN) separation membrane, wherein a final permeate stream exits a final OSN separation membrane, the final permeate stream comprising aldehydes, heavies, organophosphorous ligand, and rhodium, wherein the rhodium concentration in the final permeate stream is lower than the rhodium concentration in the tails stream; and
(c) incinerating the final permeate stream on-site to create a rhodium-containing ash. In some embodiments, the process uses one OSN separation membrane. In such embodiments, the final permeate stream is the permeate stream exiting the OSN separation membrane.

In some embodiments, the process uses at least two OSN separation membranes. In some such embodiments, all of the permeate stream from the first OSN separation membrane is provided to the second OSN separation membrane. In such embodiments, the final permeate stream is the permeate stream exiting the second (or last) OSN separation membrane. In some embodiments, only a portion of the permeate stream from the first OSN separation membrane is provided to the second OSN separation membrane. In some such embodiments, the final permeate stream comprises the portion of the permeate from the first OSN separation membrane that is not provided to the second OSN separation membrane and the permeate stream from the second OSN separation membrane. In some embodiments, the process uses at least two OSN separation membranes operated in parallel. In some such embodiments, the final permeate stream comprises the permeate stream from the first OSN separation membrane combined with the permeate stream from the second OSN separation membrane.

In some embodiments, at least a portion of a permeate stream from at least one OSN separation membrane is recycled through a previous OSN separation membrane (or the same OSN separation membrane).

In some embodiments, the incinerator in which the final permeate stream is incinerated to create the rhodium-containing ash is located within a ten mile radius of the product-catalyst separation zone. With the incinerator being located in close proximity to the hydroformylation process, embodiments of the present invention advantageously avoid complications that arise from storing and/or shipping rhodium-containing liquid purge. The incinerator in which the final permeate stream is incinerated to create the rhodium-containing ash is located within a five mile radius of the product-catalyst separation zone in some embodiments, within a three mile radius of the product-catalyst separation zone in some embodiments, or within a one mile radius of the product-catalyst separation zone in some embodiments. In some embodiments, the incinerator in which the final permeate stream is incinerated to create the rhodium-containing ash is located at the same manufacturing facility as the product-catalyst separation zone.

In some embodiments, the final permeate stream is incinerated within 90 days of the tails stream leaving the product-catalyst separation zone. In some embodiments, the final permeate stream is incinerated within 30 days of the tails stream leaving the product-catalyst separation zone. In some embodiments, the final permeate stream is incinerated within 10 days of the tails stream leaving the product-catalyst separation zone.

In some embodiments, a process of the present invention further comprises recovering rhodium from the rhodium-containing ash.

In some embodiments, the final permeate stream has a flash point of 55° C. or greater.

In some embodiments, the final permeate stream to be incinerated is treated prior to incineration to recover residual aldehyde product.

The use of the at least one OSN separation membrane provides a final permeate stream comprising aldehydes, heavies and importantly a lower rhodium content relative to the tails stream from the product-catalyst separation zone. This advantageously allows the heavies concentration in the hydroformylation reaction zone to be controlled, reduces the amount of rhodium removed from the reaction zone, and minimizes the rhodium inventory requirement of the hydroformylation process.

Incinerating the final permeate stream on-site allows virtually all of the rhodium in the tails stream to be captured and subsequently submitted for precious metal recovery (PMR). In addition, the rhodium-containing ash is easier to ship, and less expensive to ship, than a large volume of liquid, which should greatly reduce the time and total costs associated with PMR. The rhodium-containing ash is more homogeneous than large containers of fluids stored for prolonged periods and makes quantifying the precious metal (rhodium) content easier. By sizing the incinerator appropriately (e.g., continuous ashing of the final permeate stream from the tails stream based on the rate of heavies formation) and then promptly shipping the ash to the PMR facility, the amount of precious metal residing outside of the hydroformylation process at any given time is reduced. The ash is a convenient starting point for rhodium trichloride production and subsequent hydroformylation catalyst precursor manufacture.

In some embodiments, processes of the present invention allow the heavies concentration within the hydroformylation process to be controlled at a desired level, assures that a minimal amount of precious metal (e.g., rhodium) is lost from the system, and reduces precious metal inventory costs.

Turning now to starting materials used in hydroformylation, hydrogen and carbon monoxide may be obtained from any suitable source, including petroleum cracking and refinery operations.

Syngas (from synthesis gas) is the name given to a gas mixture that contains varying amounts of CO and H₂. Production methods are well known. Hydrogen and CO typically are the main components of syngas, but syngas may contain CO₂ and inert gases such as N₂ and Ar. The molar ratio of H₂ to CO varies greatly but generally ranges from 1:100 to 100:1 and preferably between 1:10 and 10:1. Syngas is commercially available and is often used as a fuel source or as an intermediate for the production of other chemicals. The most preferred H₂:CO molar ratio for chemical production is between 3:1 and 1:3 and usually is targeted to be between about 1:2 and 2:1 for most hydroformylation applications. Syngas mixtures are a preferred source of hydrogen and CO.

In some embodiments, the olefin starting material reactants comprise one or more C₆ to C₂₂ olefins. In some embodiments, the olefin starting material reactants that may be employed in the hydroformylation processes of the present invention comprise branched internal olefin mixtures, such as may be obtained from the oligomerization of butene, isobutene, etc. In some embodiments, a stream comprising mixed octenes derived from the dimerization of butenes is employed; such mixtures may be produced, for example, by the Dimersol™ process from Axens (Institut Français du Petrole, Review, Vol. 37, No 5, September-October 1982, p 639) or the Octol™ process from Huls A G (Hydrocarbon Processing, February 1992, p 45-46). In another embodiment, so called dimeric, trimeric or tetrameric propylene mixtures, as disclosed, for example, in U.S. Pat. Nos. 4,518,809 and 4,528,403 may be employed. In another embodiment the olefin mixtures employed in the process of the invention comprise ≥C₆ linear alpha olefins derived from ethylene oligomerization. In another embodiment the olefin mixtures employed in the process of the invention comprise olefins derived from a Fischer-Tropsch process.

A solvent advantageously is employed in the hydroformylation process. Any suitable solvent that does not unduly interfere with the hydroformylation process can be used. By way of illustration, suitable solvents for rhodium catalyzed hydroformylation processes include those disclosed, for example, in U.S. Pat. Nos. 3,527,809; 4,148,830; 5,312,996; and 5,929,289. Non-limiting examples of suitable solvents include saturated hydrocarbons (alkanes), aromatic hydrocarbons, ethers, aldehydes, ketones, nitriles, alcohols, esters, and aldehyde condensation products. Specific examples of solvents include: tetraglyme, pentanes, cyclohexane, heptanes, benzene, xylene, toluene, diethyl ether, tetrahydrofuran, butyraldehyde, and benzonitrile. The organic solvent may also contain dissolved water up to the saturation limit. Illustrative preferred solvents include ketones (e.g. acetone and methylethyl ketone), esters (e.g. ethyl acetate, di-2-ethylhexyl phthalate, 2,2,4-trimethyl-1,3-pentanediol monoisobutyrate), hydrocarbons (e.g. toluene), nitrohydrocarbons (e.g., nitrobenzene), ethers (e.g., tetrahydrofuran (THF)) and sulfolane. In rhodium catalyzed hydroformylation processes, it may be preferred to employ, as a primary solvent, aldehyde compounds corresponding to the aldehyde products desired to be produced and/or higher boiling aldehyde liquid condensation by-products, for example, as might be produced in situ during the hydroformylation process, as described for example in U.S. Pat. Nos. 4,148,830 and 4,247,486. The primary solvent will normally eventually comprise both aldehyde products and higher boiling aldehyde liquid condensation by-products (“heavies”), due to the nature of the continuous process. The amount of solvent is not especially critical and need only be sufficient to provide the reaction medium with the desired amount of transition metal concentration. Typically, the amount of solvent ranges from about 5 percent to about 95 percent by weight, based on the total weight of the reaction fluid. Mixtures of solvents may be employed.

The hydroformylation process also uses an organophosphorus ligand. The organophosphorous compounds that may serve as the ligand of the rhodium-complex catalyst and/or free ligand may be of the achiral (optically inactive) or chiral (optically active) type and are well known in the art. Achiral organophosphorous ligands are preferred.

Among the organophosphorous ligands that may serve as the ligand of the rhodium complex catalyst are monoorganophosphite, diorganophosphite, triorganophosphite, organopolyphosphite, triarylphosphines, polydentate phosphine compounds and mixtures thereof. Such organophosphorous ligands and/or methods for their preparation are well known in the art.

Representative monoorganophosphites may include those having the formula:

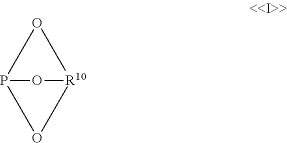

wherein R¹⁰ represents a substituted or unsubstituted trivalent hydrocarbon radical containing from 4 to 40 carbon atoms or greater, such as trivalent acyclic and trivalent cyclic radicals, e.g., trivalent alkylene radicals such as those derived from 1,2,2-trimethylolpropane and the like, or trivalent cycloalkylene radicals such as those derived from 1,3,5-trihydroxycyclohexane, and the like. Such monoorganophosphites may be found described in greater detail, for example, in U.S. Pat. No. 4,567,306.

Representative diorganophosphites may include those having the formula:

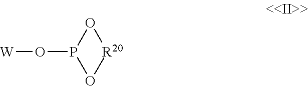

wherein R²⁰ represents a substituted or unsubstituted divalent hydrocarbon radical containing from 4 to 40 carbon atoms or greater and W represents a substituted or unsubstituted monovalent hydrocarbon radical containing from 1 to 18 carbon atoms or greater.

Representative substituted and unsubstituted monovalent hydrocarbon radicals represented by W in the above Formula (II) include alkyl and aryl radicals, while representative substituted and unsubstituted divalent hydrocarbon radicals represented by R²⁰ include divalent acyclic radicals and divalent aromatic radicals. Illustrative divalent acyclic radicals include, for example, alkylene, alkylene-oxy-alkylene, alkylene-S-alkylene, cycloalkylene radicals, and, alkylene-NR²⁴-alkylene wherein R²⁴ is hydrogen or a substituted or unsubstituted monovalent hydrocarbon radical, e.g., an alkyl radical having 1 to 4 carbon atoms. The more preferred divalent acyclic radicals are the divalent alkylene radicals such as disclosed more fully, for example, in U.S. Pat. Nos. 3,415,906 and 4,567,302 and the like. Illustrative divalent aromatic radicals include, for example, arylene, bisarylene, arylene-alkylene, arylene-alkylene-arylene, arylene-oxy-arylene, arylene-NR²⁴-arylene wherein R²⁴ is as defined above, arylene-S-arylene, and arylene-S-alkylene, and the like. More preferably R²⁰ is a divalent aromatic radical such as disclosed more fully, for example, in U.S. Pat. Nos. 4,599,206, 4,717,775, 4,835,299, and the like.

Representative of a more preferred class of diorganophosphites are those of the formula:

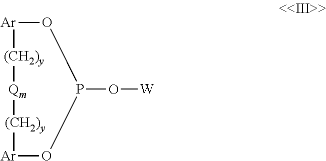

wherein W is as defined above, each Ar is the same or different and represents a substituted or unsubstituted aryl radical, each y is the same or different and is a value of 0 or 1, Q represents a divalent bridging group selected from —C(R³³)₂—, —O—, —S—, —NR²⁴—, Si(R³⁵)₂ and —CO—, wherein each R³³ is the same or different and represents hydrogen, an alkyl radical having from 1 to 12 carbon atoms, phenyl, tolyl, and anisyl, R²⁴ is as defined above, each R³⁵ is the same or different and represents hydrogen or a methyl radical, and m has a value of 0 or 1. Such diorganophosphites are described in greater detail, for example, in U.S. Pat. Nos. 4,599,206, 4,717,775, and 4,835,299.

Representative triorganophosphites may include those having the formula:

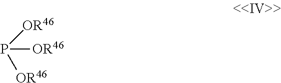

wherein each R⁴⁶ is the same or different and is a substituted or unsubstituted monovalent hydrocarbon radical e.g., an alkyl, cycloalkyl, aryl, alkaryl and aralkyl radicals that may contain from 1 to 24 carbon atoms. Illustrative triorganophosphites include, for example, trialkyl phosphites, dialkylaryl phosphites, alkyldiaryl phosphites, triaryl phosphites, and the like, such as, for example, trimethyl phosphite, triethyl phosphite, butyldiethyl phosphite, tri-n-propyl phosphite, tri-n-butyl phosphite, tri-2-ethylhexyl phosphite, tri-n-octyl phosphite, tri-n-dodecyl phosphite, dimethylphenyl phosphite, diethylphenyl phosphite, methyldiphenyl phosphite, ethyldiphenyl phosphite, triphenyl phosphite, trinaphthyl phosphite, bis(3,6,8-tri-t-butyl-2-naphthyl)methylphosphite, bis(3,6,8-tri-t-butyl-2-naphthyl)cyclohexylphosphite, tris(3,6-di-t-butyl-2-naphthyl)phosphite, bis(3,6,8-tri-t-butyl-2-naphthyl)(4-biphenyl)phosphite, bis(3,6,8-tri-t-butyl-2-naphthyl)phenylphosphite, bis(3,6,8-tri-t-butyl-2-naphthyl)(4-benzoylphenyl)phosphite, bis(3,6,8-tri-t-butyl-2-naphthyl)(4-sulfonylphenyl)phosphite, and the like. A preferred triorganophosphite is tris(2,4-di-t-butylphenyl)phosphite. Such triorganophosphites are described in greater detail, for example, in U.S. Pat. Nos. 3,527,809 and 4,717,775.

Representative organopolyphosphites contain two or more tertiary (trivalent) phosphorus atoms and may include those having the formula:

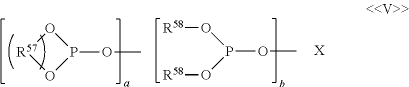

wherein X represents a substituted or unsubstituted n-valent organic bridging radical containing from 2 to 40 carbon atoms, each R⁵⁷ is the same or different and represents a divalent organic radical containing from 4 to 40 carbon atoms, each R⁵⁸ is the same or different and represents a substituted or unsubstituted monovalent hydrocarbon radical containing from 1 to 24 carbon atoms, a and b can be the same or different and each have a value of 0 to 6, with the proviso that the sum of a+b is 2 to 6 and n equals a+b. When “a” has a value of 2 or more, each R⁵⁷ radical may be the same or different. Each R⁵⁸ radical may also be the same or different in any given compound.

Representative n-valent (preferably divalent) organic bridging radicals represented by X and representative divalent organic radicals represented by R⁵⁷ above, include both acyclic radicals and aromatic radicals, such as alkylene, alkylene-Qₘ-alkylene, cycloalkylene, arylene, bisarylene, arylene-alkylene, and arylene-(CH₂)_{y}-Qₘ-(CH₂)_{y}-arylene radicals, and the like, wherein each Q, y and m are as defined above in Formula (III). The more preferred acyclic radicals represented by X and R⁵⁷ above are divalent alkylene radicals, while the more preferred aromatic radicals represented by X and R⁵⁷ above are divalent arylene and bisarylene radicals, such as disclosed more fully, for example, in U.S. Pat. Nos. 4,769,498; 4,774,361: 4,885,401; 5,179,055; 5,113,022; 5,202,297; 5,235,113; 5,264,616; 5,364,950 and 5,527,950. Representative preferred monovalent hydrocarbon radicals represented by each R⁵⁸ radical above include alkyl and aromatic radicals.

Illustrative preferred organopolyphosphites may include bisphosphites such as those of Formulas (VI) to (VIII) below:

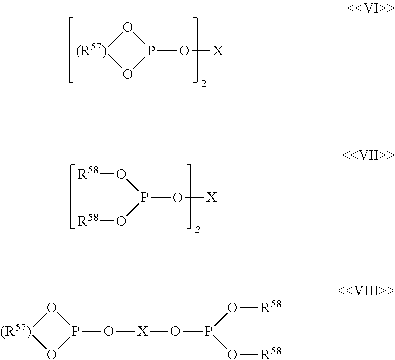

wherein each R⁵⁷, R⁵⁸ and X of Formulas (VI) to (VIII) are the same as defined above for Formula (V). Preferably each R⁵⁷ and X represents a divalent hydrocarbon radical selected from alkylene, arylene, arylene-alkylene-arylene, and bisarylene, while each R⁵⁸ radical represents a monovalent hydrocarbon radical selected from alkyl and aryl radicals.

Organophosphite ligands of such Formulas (V) to (VIII) may be found disclosed, for example, in U.S. Pat. Nos. 4,668,651; 4,748,261; 4,769,498; 4,774,361; 4,885,401; 5,113,022; 5,179,055; 5,202,297; 5,235,113; 5,254,741; 5,264,616; 5,312,996; 5,364,950; and 5,391,801.

R¹⁰, R²⁰, R⁴⁶, R⁵⁷, R⁵⁸, Ar, Q, X, m, and y in Formulas (VI) to (VIII) are as defined above. Most preferably X represents a divalent aryl-(CH₂)_{y}-(Q)ₘ-(CH₂)_{y}-aryl radical wherein each y individually has a value of 0 or 1; m has a value of 0 or 1 and Q is —O—, —S— or —C(R³⁵)₂— where each R³⁵ is the same or different and represents hydrogen or a methyl radical. More preferably each alkyl radical of the above defined ^{R58} groups may contain from 1 to 24 carbon atoms and each aryl radical of the above-defined Ar, X, R⁵⁷ and R⁵⁸ groups of the above Formulas (VI) to (VII) may contain from 6 to 18 carbon atoms and said radicals may be the same or different, while the preferred alkylene radicals of X may contain from 2 to 18 carbon atoms and the preferred alkylene radicals of R⁵⁷ may contain from 5 to 18 carbon atoms. In addition, preferably the divalent Ar radicals and divalent aryl radicals of X of the above formulas are phenylene radicals in which the bridging group represented by —(CH₂)_{y}-(Q)ₘ-(CH₂)_{y}— is bonded to said phenylene radicals in positions that are ortho to the oxygen atoms of the formulas that connect the phenylene radicals to their phosphorus atom of the formulae. It is also preferred that any substituent radical when present on such phenylene radicals be bonded in the para and/or ortho position of the phenylene radicals in relation to the oxygen atom that bonds the given substituted phenylene radical to its phosphorus atom.

Any of the R¹⁰, R²⁰, R⁵⁷, R⁵⁸, W, X, Q and Ar radicals of such organophosphites of Formulas (I) to (VIII) above may be substituted if desired, with any suitable substituent containing from 1 to 30 carbon atoms that does not unduly adversely affect the desired result of the process of this invention. Substituents that may be on said radicals in addition to corresponding hydrocarbon radicals such as alkyl, aryl, aralkyl, alkaryl and cyclohexyl substituents, may include for example silyl radicals such as —Si(R³⁵)₃; amino radicals such as —N(R¹⁵)₂; phosphine radicals such as -aryl-P(R¹⁵)₂; acyl radicals such as —C(O)R¹⁵ acyloxy radicals such as —OC(O)R¹⁵; amido radicals such as —CON(R¹⁵)₂ and —N(R¹⁵)COR¹⁵; sulfonyl radicals such as —SO₂R¹⁵, alkoxy radicals such as —OR¹⁵; sulfinyl radicals such as —SOR¹⁵, phosphonyl radicals such as —P(O)(R¹⁵)₂, as well as halo, nitro, cyano, trifluoromethyl, hydroxy radicals, and the like, wherein each R¹⁵ radical individually represents the same or different monovalent hydrocarbon radical having from 1 to 18 carbon atoms (e.g., alkyl, aryl, aralkyl, alkaryl and cyclohexyl radicals), with the proviso that in amino substituents such as —N(R¹⁵)₂ each R¹⁵ taken together can also represent a divalent bridging group that forms a heterocyclic radical with the nitrogen atom, and in amido substituents such as —C(O)N(R¹⁵)₂ and —N(R¹⁵)COR¹⁵ each R¹⁵ bonded to N can also be hydrogen. Any of the substituted or unsubstituted hydrocarbon radicals groups that make up a particular given organophosphite may be the same or different.

More specifically illustrative substituents include primary, secondary and tertiary alkyl radicals such as methyl, ethyl, n-propyl, isopropyl, butyl, sec-butyl, t-butyl, neo-pentyl, n-hexyl, amyl, sec-amyl, t-amyl, iso-octyl, decyl, octadecyl, and the like; aryl radicals such as phenyl, naphthyl and the like; aralkyl radicals such as benzyl, phenylethyl, triphenylmethyl, and the like; alkaryl radicals such as tolyl, xylyl, and the like; alicyclic radicals such as cyclopentyl, cyclohexyl, 1-methylcyclohexyl, cyclooctyl, cyclohexylethyl, and the like; alkoxy radicals such as methoxy, ethoxy, propoxy, t-butoxy, —OCH₂CH₂ OCH₃, —O(CH₂CH₂)₂OCH₃, —O(CH₂CH₂)₃OCH₃, and the like; aryloxy radicals such as phenoxy and the like; as well as silyl radicals such as —Si(CH₃)₃, —Si(OCH₃)₃, —Si(C₃H₇)₃, and the like; amino radicals such as —NH₂, —N(CH₃)₂, —NHCH₃, —NH(C₂H₅), and the like; arylphosphine radicals such as —P(C₆H₅)₂, and the like; acyl radicals such as —C(O)CH₃, —C(O)C₂H₅, —C(O)C₆H₅, and the like; carbonyloxy radicals such as —C(O)OCH₃ and the like; oxycarbonyl radicals such as —O(CO)C₆H₅, and the like; amido radicals such as —CONH₂, —CON(CH₃)₂, —NHC(O)CH₃, and the like; sulfonyl radicals such as —S(O)₂C₂H₅ and the like; sulfinyl radicals such as —S(O)CH₃ and the like; sulfidyl radicals such as —SCH₃, —SC₂H₅, —SC₆H₅, and the like; phosphonyl radicals such as —P(O)(C₆H₅)₂, —P(O)(CH₃)₂, —P(O)(C₂H₅)₂, —P(O)(C₃H₇)₂, —P(O)(C₄H₉)₂, —P(O)(C₆H₁₃)₂, —P(O)CH₃ (C₆H₅), —P(O)(H)(C₆H₅), and the like.

Specific illustrative examples of such organophosphite ligands include the following: 2-t-butyl-4-methoxyphenyl(3,3′-di-t-butyl-5,5′-dimethoxy-1,1′-biphenyl-2,2′-diyl)phosphite, methyl(3,3′-di-t-butyl-5,5′-dimethoxy-1,1′-biphenyl-2,2′-diyl)phosphite, 6,6′-[[4,4′-bis(1,1-dimethylethyl)-[1,1′-binaphthyl]-2,2′-diyl]bis(oxy)]bis-dibenzo[d,f][1,3,2]-dioxaphosphepin, 6,6′-[[3,3′-bis(1,1-dimethylethyl)-5,5′-dimethoxy-[1,1′-biphenyl]-2,2′-diyl]bis(oxy)]bis-dibenzo[d,f][1,3,2]dioxaphosphepin, 6,6′-[[3,3′,5,5′-tetrakis(1,1-dimethylpropyl)-[1,1′-biphenyl]-2,2′-diyl]bis(oxy)]bis-dibenzo[d,f][1,3,2]dioxaphosphepin, 6,6′-[[3,3′,5,5′-tetrakis(1,1-dimethylethyl)-1,1′-biphenyl]-2,2′-diyl]bis(oxy)]bis-dibenzo[d,f][1,3,2]-dioxaphosphepin, (2R,4R)-di [2,2′-(3,3′,5,5′-tetrakis-tert-amyl-1,1-biphenyl)]-2,4-pentyldiphosphite, (2R,4R)-di[2,2′-(3,3′,5,5′-tetrakis-tert-butyl-1,1-biphenyl)]-2,4-pentyldiphosphite, (2R,4R)-di[2,2′-(3,3′-di-amyl-5,5′-dimethoxy-1,1′-biphenyl)]2,4-pentyldiphosphite, (2R,4R)-di[2,2′-(3,3′-di-tert-butyl-5,5′-dimethyl-1,1′-biphenyl)]-2,4-pentyldiphosphite, (2R,4R)-di[2,2′-(3,3′-di-tert-butyl-5,5′-diethoxy-1,1′-biphenyl)]-2,4-pentyldiphosphite, (2R,4R),di[2,2′(3,3′di-tert,butyl-5,5-diethyl-1,1-biphenyl)]-2,4-pentyldiphosphite, (2R,4R)di[2,2′-(3,3′-di-tert-butyl-5,5′-dimethoxy-1,1′-biphenyl)]-2,4-pentyldiphosphite, 6-[[2′-[(4,6-bis(1,1-dimethylethyl)-1,3,2-benzodioxaphosphol-2-yl)oxy]-3,3′-bis(1,1-dimethylethyl)-5,5′-dimethoxy[1,1′-biphenyl]-2-yl]oxy]-4,8-bis(1,1-dimethylethyl)-2,10-dimethoxydibenzo [d,f][1,3,2]dioxaphosphepin, 6-[[2′-[1,3,2-benzodioxaphosphol-2-yl)oxy]-3,3′-bis(1,1-dimethylethyl)-5,5′-dimethoxyl[1,1′-biphenyl]-2-yl]oxy]-4,8-bis(1,1-dimethylethyl)-2,10-dimethoxydibenzo[d,f][1,3,2]dioxaphosphepin, 6-[[2′-[(5,5-dimethyl-1,3,2-dioxaphosphorinan-2-yl) oxy]-3,3′-bis(1,1-dimethylethyl)-5,5′-dimethoxy[1,1′-biphenyl]-2-yl]oxy]-4,8-bis(1,1-dimethylethyl)-2,10-dimethoxydibenzo[d,f][1,3,2]dioxaphosphepin, 2′-[[4,8-bis(1,1-dimethylethyl)-2,10-dimethoxydibenzo[d,f][1,3,2]-dioxaphosphepin-6-yl]oxy]-3,3′-bis (1,1-dimethylethyl)-5,5′-dimethoxy[1,1′-biphenyl]-2-yl bis(4-hexylphenyl)ester of phosphorous acid, 2-[[2-[[4,8,-bis(1,1-dimethylethyl), 2,10-dimethoxydibenzo-[d,f][1,3,2]dioxophosphepin-6-yl]oxy]-3-(1,1-dimethylethyl)-5-methoxyphenyl]methyl]-4-methoxy, 6-(1,1-dimethylethyl)phenyl, diphenyl ester of phosphorous acid, 3-methoxy-1,3-cyclohexamethylene tetrakis[3,6-bis(1,1-dimethylethyl)-naphthalenyl]ester of phosphorous acid, 2,5-bis(1,1-dimethylethyl)-1,4-phenylene tetrakis[2,4-bis(1,1-dimethylethyl)phenyl]ester of phosphorous acid, methylenedi-2,1-phenylene tetrakis[2,4-bis(1,1-dimethylethyl)phenyl]ester of phosphorous acid, and [1,1′-biphenyl]-2,2′-diyl tetrakis[2-(1,1-dimethylethyl)-4-methoxyphenyl]ester of phosphorous acid.

Triarylphosphines that may serve as the ligand of the invention comprise any organic compound comprising at least one phosphorus atom covalently bound to three aryl or arylalkyl radicals, or combinations thereof. A mixture of triarylphosphine ligands may also be employed. Representative organomonophosphines include those having the formula:

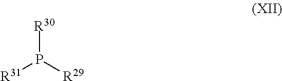

wherein each R²⁹, R³⁰ and R³¹ may be the same or different and represent a substituted or unsubstituted aryl radical containing from 4 to 40 carbon atoms or greater. Such triarylphosphines may be found described in greater detail, for example, in U.S. Pat. No. 3,527,809, the disclosure of which is incorporated herein by reference. Illustrative triarylphosphine ligands are triphenylphosphine, trinaphthylphine, tritolylphosphine, tri(p-biphenyl)phosphine, tri(p-methoxyphenyl) phosphine, tri(m-chlorophenyl)-phosphine, p-N,N-dimethylaminophenyl bis-phenyl phosphine, and the like. Triphenyl phosphine, i.e., the compound of Formula I wherein each R²⁹, R³⁰ and R³¹ is phenyl, is an example of a preferred organomonophosphine ligand.

The polydentate phosphines that may serve as the ligand of the invention may comprise organic compounds containing two or more phosphorous atoms. Specific examples include 2,2′-Bis(diphenylphosphinomethyl)-1,1′-biphenyl (BISBI) and substituted variation thereof, such as is described in WO1989006653A1.

The polydentate phosphines that may serve as the ligand of the invention comprise tetraphosphine compounds of the Formula XIII:

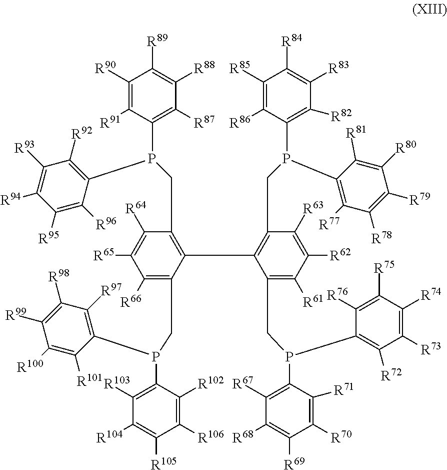

Wherein each P is a phosphorous atom, and each of R⁶¹-R¹⁰⁵ are independently hydrogen, a C₁ to C₈ alkyl group, an aryl group, an alkaryl group, or a halogen. In a preferred embodiment, each of R⁶¹-R¹⁰⁵ is hydrogen. Such compounds are disclosed, for example, in U.S. Pat. No. 7,531,698.

In one embodiment, a mixture of ligands is employed. In one embodiment, a mixture comprised of a tetradentate phosphine and a triarylphosphine is employed.

The catalyst of this invention comprises rhodium and an organophosphorous ligand. The rhodium can be introduced to the liquid body as a precursor form which is converted in situ into the catalyst. Examples of such precursor form are rhodium carbonyl triphenylphosphine acetylacetonate, Rh₂O₃, Rh₄(CO)₁₂, Rh₆(CO)₁₆, and rhodium dicarbonyl acetylacetonate. Both the catalyst compounds which will provide active species in the reaction medium and their preparation are known by the art, see Brown et al., Journal of the Chemical Society, 1970, pp. 2753-2764.

In ultimate terms the rhodium concentration in the liquid body can range from about 10 ppm to about 1200 ppm of rhodium calculated as free metal. The amount of organophosphorous ligand in the liquid body may vary depending on the nature of the ligand. In one embodiment, a triorganophosphite may be used in the range of about 0.2-8 percent by weight, based on the weight of the total reaction mixture. In one embodiment, a monodentate phosphine may be used in the range of 5 percent to 15 percent by weight, based on the weight of the total reaction mixture. In another embodiment, a chelating ligand such as an organopolyphosphite is used at a concentration of about 1-5 molar equivalents relative to rhodium. In another embodiment a polyphosphine is present in the range of about 1 to 10 moles per mole of rhodium. In another embodiment, a mixture comprising a polyphosphine in the range of 1-5 molar equivalents relative to rhodium and 2-15 wt. % of triarylphosphine is employed. In another embodiment a tetraphosphine is present in the range of about 1 to 10 moles per mole of rhodium. In another embodiment, a mixture comprising a tetraphosphine in the range of 1-5 molar equivalents relative to rhodium and 2-15 wt. % of triarylphosphine is employed.

The rhodium complex catalysts may be in homogeneous or heterogeneous form. For instance, preformed rhodium hydrido-carbonyl-phosphine ligand catalysts may be prepared and introduced into a hydroformylation reaction mixture. More preferably, the rhodium-phosphine ligand complex catalysts can be derived from a rhodium catalyst precursor that may be introduced into the reaction medium for in situ formation of the active catalyst. For example, rhodium catalyst precursors such as rhodium dicarbonyl acetylacetonate, Rh₂O₃, Rh₄(CO)₁₂, Rh₆(CO)₁₆, Rh(NO₃)₃ and the like may be introduced into the reaction mixture along with the organophosphorous ligand for the in situ formation of the active catalyst. In one embodiment, rhodium dicarbonyl acetylacetonate is employed as a rhodium precursor and reacted in the presence of a solvent with triarylphosphine to form a catalytic rhodium-triarylphosphine ligand complex precursor that is introduced into the reactor along with excess (free) triarylphosphine for the in situ formation of the active catalyst. In one embodiment, rhodium dicarbonyl acetylacetonate is employed as a rhodium precursor and reacted in the presence of a solvent with an organopolyphosphite to form a catalytic rhodium-organopolyphosphite ligand complex precursor that is introduced into the reactor along with excess (free) organopolyphosphite for the in situ formation of the active catalyst. In any event, it is sufficient that carbon monoxide, hydrogen and the organophosphorous ligands are all capable of complexing with the metal and that an active metal-ligand catalyst is present in the reaction mixture under the conditions used in the hydroformylation reaction. Carbonyl and organophosphorous ligands may be complexed to the rhodium either prior to or in situ during the hydroformylation process.

By way of illustration, a preferred catalyst precursor composition consists essentially of a solubilized rhodium complex precursor, a solvent and excess organophosphorous ligand. In most cases, the organophosphorous ligand readily replaces one of the carbonyl ligands of the rhodium acetylacetonate complex precursor as witnessed by the evolution of carbon monoxide gas.

Accordingly, the rhodium-ligand complex catalysts advantageously comprise the rhodium complexed with carbon monoxide and organophosphorus ligand, wherein at least one organophosphorous molecule is bonded (complexed) to the metal. The catalyst additionally comprises rhodium complexed with carbon monoxide and polydentate organophosphorous compounds such as a organopolyphosphites or tetradentate phosphines in a chelated and/or non-chelated fashion.

In one embodiment, mixtures of rhodium complexes are employed. The amount of rhodium complex catalyst present in the reaction fluid need only be that minimum amount necessary to produce the desired production rate. In general, rhodium concentrations in the range of from 10 ppmw to 1000 ppmw, calculated as free metal in the reaction medium, should be sufficient for most processes, while it is generally preferred to employ from 10 to 500 ppmw of metal, and more preferably from 25 to 350 ppmw of rhodium.

In addition to the rhodium complex catalyst, free organophosphorous ligand (i.e., organophosphorous ligand that is not complexed with the metal) will also be present in the reaction medium. The significance of free ligand is taught in U.S. Pat. No. 3,527,809, GB 1,338,225, and Brown et al., supra., pages 2759 and 2761. By way of example, a hydroformylation process of this invention which employs triarylphosphine may involve from 5 to 15 wt. % or higher of free triarylphosphine in the reaction medium. In another embodiment, a hydroformylation process of this invention which employs an organopolyphosphite ligand will also contain free organopolyphosphite ligand. The concentration of free organopolyphosphite ligand in the reaction fluid may range from about 0.1 to 10 moles per mole of rhodium. In another embodiment, a hydroformylation process of this invention which employs a tetradentate phosphine ligand will also contain free tetradentate phosphine ligand. The concentration of free tetradentate phosphine ligand in the reaction fluid may range from about 0.1 to 10 moles per mole of rhodium. In another embodiment, a hydroformylation process of this invention which employs a mixture of tetradentate phosphine ligand and triarylphosphine ligand will also contain both free tetradentate phosphine and triarylphosphine ligands. The concentration of free tetradentate phosphine ligand in the reaction fluid may range from about 0.1 to 10 moles per mole of rhodium, while the triarylphosphine may range from about 2 to 15 wt. %.

The hydroformylation process, and conditions for its operation, are well known. In a preferred embodiment, one or more olefins is hydroformylated in a continuous or semi-continuous fashion, with the product being separated in a product-catalyst separation zone, and the concentrated catalyst solution being recycled back into the reactors. The catalyst recycle procedure generally involves withdrawing a portion of the liquid reaction medium containing the catalyst and aldehyde product from the hydroformylation reactor, i.e., reaction zone, either continuously or intermittently, and recovering a portion of the aldehyde product therefrom by vaporization separation, in one or more stages under normal, reduced or elevated pressure as appropriate. The non-volatile effluent which has been stripped of a portion of the aldehyde product and comprises the rhodium-complex catalyst is then recycled to the reaction zone as disclosed, for example, in U.S. Pat. No. 5,288,918. Such types of recycle procedures are well known in the art and may involve the liquid recycling of the metal-organophosphorous complex catalyst fluid separated from the desired aldehyde reaction product(s), such as disclosed, for example, in U.S. Pat. No. 4,148,830 or a gas recycle procedure such as disclosed, for example, in U.S. Pat. No. 4,247,486, as well as a combination of both a liquid and gas recycle procedure if desired. The most preferred hydroformylation process comprises a continuous liquid catalyst recycle process. Suitable liquid catalyst recycle procedures are disclosed, for example, in U.S. Pat. Nos. 4,668,651; 4,774,361; 5,102,505 and 5,110,990. Condensation of the volatilized materials, and separation and further recovery thereof, e.g., by further distillation, can be carried out in any conventional manner, the crude aldehyde product can be passed on for further purification and isomer separation, if desired, and any recovered reactants, e.g., olefinic starting material and syngas, can be recycled in any desired manner to the hydroformylation zone (reactor).

In a preferred embodiment, the hydroformylation reaction fluid contains at least some amount of four main ingredients or components, i.e., the aldehyde product, a rhodium-organophosphorous ligand complex catalyst, free organophosphorous ligand, and a solvent for said catalysts and said free ligands. The hydroformylation reaction mixture compositions can and normally will contain additional ingredients such as those that have either been deliberately employed in the hydroformylation process or formed in situ during said process. Examples of such additional ingredients include unreacted olefin starting material, carbon monoxide and hydrogen gases, and in situ formed by-products, ligand degradation compounds, and high boiling liquid aldehyde condensation by-products (heavies), as well as other inert co-solvent type materials or hydrocarbon additives, if employed.

The hydroformylation reaction conditions employed may vary. For instance, the total gas pressure of hydrogen, carbon monoxide and olefin starting compound of the hydroformylation process may range from 1 to 69,000 kPa. In general, however, it is preferred that the process be operated at a total gas pressure of hydrogen, carbon monoxide and olefin starting compound of less than 14,000 kPa and more preferably less than 3,400 kPa. The minimum total pressure is limited predominantly by the amount of reactants necessary to obtain a desired rate of reaction. More specifically, the carbon monoxide partial pressure of the hydroformylation process is preferably from 1 to 6,900 kPa, and more preferably from 21 to 5,500 kPa, while the hydrogen partial pressure is preferably from 34 to 3,400 kPa and more preferably from 69 to 2,100 kPa. In general, the molar ratio of gaseous H₂:CO may range from 1:10 to 100:1 or higher, the more preferred molar ratio being from 1:10 to 10:1.

In general, the hydroformylation process may be conducted at any operable reaction temperature. Advantageously, the hydroformylation process is conducted at a reaction temperature from −25° C. to 200° C., preferably from 50° C. to 120° C.

The hydroformylation process may be carried out using one or more suitable reactors such as, for example, a continuous stirred tank reactor (CSTR) or a bubble or plug flow reactor. The optimum size and shape of the reactor will depend on the type of reactor used. The reaction zone employed may be a single vessel or may comprise two or more discrete vessels. The product-catalyst separation zone employed may be a single vessel or may comprise two or more discrete vessels.

The hydroformylation process can be conducted with recycle of unconsumed starting materials (e.g., unreacted olefins) if desired. The reaction can be conducted in a single reaction zone or in a plurality of reaction zones, and in series or in parallel. The reaction steps may be effected by the incremental addition of one of the starting materials to the other. Also, the reaction steps can be combined by the joint addition of the starting materials. The starting materials may be added to each or all of the reaction zones in series. When complete conversion is not desired or not obtainable, the starting materials can be separated from the product, for example by distillation, and the starting materials then recycled back into the reaction zone.

The hydroformylation process may be conducted in either glass lined, stainless steel or similar type reaction equipment. The reaction zone may be fitted with one or more internal and/or external heat exchanger(s) in order to control undue temperature fluctuations, or to prevent any possible “runaway” reaction temperatures.

The hydroformylation process of this invention may be conducted in one or more steps or stages. The exact number of reaction steps or stages will be governed by the best compromise between capital costs and achieving high catalyst selectivity, activity, lifetime and ease of operability, as well as the intrinsic reactivity of the starting materials in question and the stability of the starting materials and the desired reaction product to the reaction conditions.

In one embodiment, the hydroformylation process useful in this invention may be carried out in a multistaged reactor such as described, for example, in U.S. Pat. No. 5,728,893. Such multistaged reactors can be designed with internal, physical barriers that create more than one theoretical reactive stage per vessel.

In one embodiment, the aldehyde product mixtures, produced by any suitable method, may be separated from the other components of the crude reaction mixtures in a product-catalyst separation zone comprising, for example, solvent extraction, crystallization, distillation, vaporization, wiped film evaporation, falling film evaporation, phase separation, filtration, or any combination thereof. It may be desired to remove the aldehyde products from the crude reaction mixture as they are formed through the use of trapping agents as described in PCT Publication No. WO 1988/008835. In some embodiments, the aldehyde products are removed from the crude reaction mixture using a strip gas vaporizer.

As indicated above, desired aldehydes may be recovered from the reaction mixtures. For example, the recovery techniques disclosed in U.S. Pat. Nos. 4,148,830 and 4,247,486 can be used. For instance, in a continuous liquid catalyst recycle process the portion of the liquid reaction mixture (containing aldehyde product, catalyst, etc.), i.e., reaction fluid, removed from the reaction zone can be passed to a product-catalyst separation zone, e.g., vaporizer/separator, wherein the desired aldehyde product can be separated via distillation, in one or more stages, under normal, reduced or elevated pressure, from the liquid reaction fluid, condensed and collected in a product receiver, and further purified if desired. The remaining non-volatilized catalyst containing liquid reaction mixture may then be recycled back to the reactor as may, if desired, any other volatile materials, e.g., unreacted olefin, together with any hydrogen and carbon monoxide dissolved in the liquid reaction after separation thereof from the condensed aldehyde product, e.g., by distillation in any conventional manner.

More particularly, distillation and separation of the desired aldehyde product from the metal-organophosphorous complex catalyst containing reaction fluid may take place at any suitable temperature desired. In general, it is preferred that such distillation take place at relatively low temperatures, such as below 170° C., and more preferably at a temperature in the range of from 50° C. to 165° C. In one embodiment, such aldehyde distillation takes place in the presence of flowing gas which becomes saturated with the product and helps carry it to a condenser, thereby allowing the aldehydes to be collected as a liquid. Such strip gas vaporizers are described for example in U.S. Pat. No. 8,404,903 and US 2014-62087572. In another embodiment, the separation may take place under vacuum which allows high boiling aldehydes (e.g. C₇ or greater) to be volatilized at lower temperatures.

In some embodiments, the aldehyde product is comprised of a mixture of normal and branched ≥C₇ aldehydes.

In embodiments of the present invention, at least a portion of the tails stream from the product-catalyst separation zone is provided to at least one organic solvent nanofiltration (OSN) separation membrane. In some embodiments, the entire tails stream from the product-catalyst separation zone is provided to at least one OSN separation membrane.

As noted above, OSN separation membranes suitable for use in some embodiments of the present invention include a surface active layer that comprises, for example and without limitation, polyldimethylsiloxane (PDMS), polyimides, polyamides, polyetheretherketone (PEEK), polypropylene, and the like. In some embodiments, the OSN separation membrane may further be comprised of a support material to which one or more surface active layers have been applied. Non-limiting examples of such membranes are known and are described for example in U.S. Pat. Nos. 5,430,194, 5,681,473, 6,252,123, and 9,828,656. Examples of OSN separation membranes that can be used in some embodiments of the present invention are commercially available from Borsig Membrane Technology.

In some embodiments, multiple OSN separation membranes may be used. As noted elsewhere, in some embodiments, at least two OSN separation membranes are used. In some embodiments, two OSN separation membranes are used in series (e.g., a permeate stream from the first OSN separation membrane is an OSN feed to the second OSN separation membrane, and the permeate stream from the second (or last) OSN separation membrane is the final permeate stream). In some embodiments, two OSN separation membranes are used in parallel (e.g., a tails stream from a product-catalyst separation zone is split with a portion being an OSN feed to a first OSN separation membrane and the other portion being an OSN feed to a second OSN separation membrane, and the permeate streams from both OSN separation membranes being combined to form the final permeate stream).

In some embodiments, the final permeate stream is treated prior to incineration to recover residual aldehyde product. Since the catalyst at this stage will not be recycled to the reaction zone, more rigorous conditions can be employed to recover the residual aldehyde without being concerned about catalyst degradation. Examples of such treatment include, but are not limited to distillation (e.g, vacuum distillation, wiped film evaporation, etc.).

The final permeate stream is provided to an incinerator to generate a rhodium-containing ash. In general, any incinerator known to those having ordinary skill in the art as being useful for generating a rhodium ash from an organic reaction fluid may be used. For example, equipment which is suitable for the incineration step of processes of the present invention may be purchased from a commercial vendor. The design should include suitable means to insure that precious metal (e.g., rhodium) is not lost in the flue gas. Examples of such features include, but are not limited to, a primary combustion chamber with post-combustion cyclonic ash removal; combustion in an ash retaining tray, followed by secondary combustion and downstream quench/wash/filtration/electrostatic filtration steps prior to sending the flue gas to the exhaust stack.

The size of the incinerator can be selected based on the expected rate of generation of the final permeate stream, desired incineration frequency, safety, and other factors known to those having ordinary skill in the art.

The incinerator, in some embodiments, is located on-site with the hydroformylation process. As used herein, the term “on-site” generally means that the incinerator is in the same general location as the product-catalyst separation zone such that the final permeate stream does not need to be transported a long distance for incineration. Typically, the incinerator will be located within the same battery limits as the product-catalyst separation zone. The incinerator, in some embodiments, is located at the same manufacturing facility as the product-catalyst separation zone. In some embodiments, the incinerator is located within a ten mile radius of the product-catalyst separation zone. The incinerator in which the final permeate stream is incinerated to create the rhodium-containing ash is located within a five mile radius of the product-catalyst separation zone in some embodiments, within a three mile radius of the product-catalyst separation zone in some embodiments, or within a one mile radius of the product-catalyst separation zone in some embodiments. In some embodiments, the incinerator is connected to an OSN stage by piping and there is no isolation and/or transportation of the final permeate stream using totes, drums, rail cars, trucks, and the like.

The resulting rhodium-containing ash may then be collected and shipped to a precious metal refiner for recovery of the metal content.

With the incinerator being located in close proximity to the hydroformylation process, embodiments of the present invention advantageously avoid complications that arise from long-term storage and/or shipping of rhodium-containing liquid purge. There are a number of advantages of storing/transporting rhodium-containing ash for recovery of rhodium instead of storing/transporting a rhodium-containing purge stream from a hydroformylation process. For example, the purge stream will have a larger volume and greater mass, will have a flash point that will need to be considered, and will be a liquid necessitating different shipping precautions (e.g., avoiding leaks) relative to solids such as ash. Additionally if the purge stream fluid is stored for extended periods, precipitation of rhodium-containing solids can potentially occur, which will make both quantifying and recovering the rhodium content more difficult.

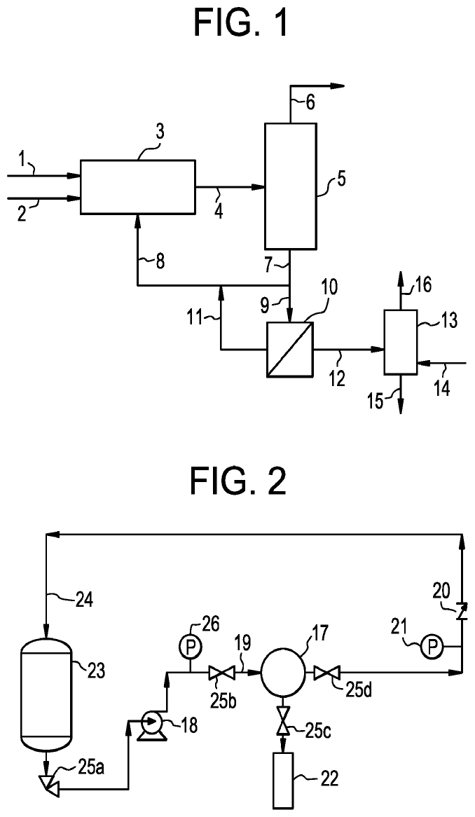

FIG. 1 is a schematic of a system for implementing some embodiments of processes of the present invention. In FIG. 1, olefin 1 and syngas 2 are fed to a reaction zone 3. A portion of the reaction fluid in the reaction zone 3 is removed via line 4 and provided to a product-catalyst separation zone 5 (e.g., a strip gas vaporizer). A crude product stream 6 is removed for further processing downstream (e.g., hydrogenation, aldolization, etc.). A tails stream 7 is removed from the product-catalyst separation zone 5. In the embodiment shown, a portion of the tails stream 7 is sent back to the reaction zone 3 via line 8 (optionally with catalyst treatment processes, not shown) while in other embodiments, all of the tails stream 7 is provided to the OSN stage 10. The remaining portion of tails stream 7 is diverted via stream 9 to at least one OSN separation membrane represented as OSN stage 10. The retentate 11 is returned to the reaction zone 3 via line 8 or may be delivered elsewhere in the reaction system. The final permeate stream 12 is sent to an incinerator 13 (optionally via a surge or day tank, not shown) wherein the liquid stream is incinerated with an oxidant 14, typically an O₂-containing gas such as air, to generate the precious metal containing ash 15 and gaseous effluent 16 comprising mostly of nitrogen, CO₂, and water vapor. Various other systems can be used to implement embodiments of the present invention based on the teachings herein.

Analytical techniques for measuring the concentration of organophosphorous compounds in catalyst solutions are well known to the skilled person and include High Performance Liquid Chromatography (HPLC) and Gas Chromatography (GC). HPLC is typically preferred.

Analytical techniques for measuring catalytic metal concentrations are well known to the skilled person, and include atomic absorption (AA), inductively coupled plasma (ICP) and X-ray fluorescence (XRF). In embodiments of the present invention, atomic absorption (AA) is used to measure catalytic metal concentration in liquids as known to those of ordinary skill in the art. Catalytic metal concentration in ash following incineration may also be determined by XRF or ICP. In one embodiment, the concentration of metal in the ash following incineration is measured by both ICP and XRF.

Analytical techniques for measuring the concentration of heavies in the catalyst solution are well known to the skilled person and include GC. The viscosity of the fluid may also be measured and a correlation between heavies concentration and viscosity established. GC is typically preferred.

Some embodiments of the present invention will now be described in more detail in the following Examples.

### EXAMPLES

All parts and percentages in the following examples are by weight unless otherwise indicated. Pressures in the following examples are given as pounds per square inch gauge unless otherwise indicated. All manipulations such as preparation of catalyst solutions, storage of OSN feed and collection of OSN permeate are done under inert atmosphere unless otherwise indicated. OSN separation membranes are a silicone-based composite membrane from Borsig Membrane Technologies (o-NF1 or o-NF2).

The hydroformylation catalyst is comprised of rhodium (dicarbonyl) acetylacetonate and one of the organophosphorous compounds shown below:

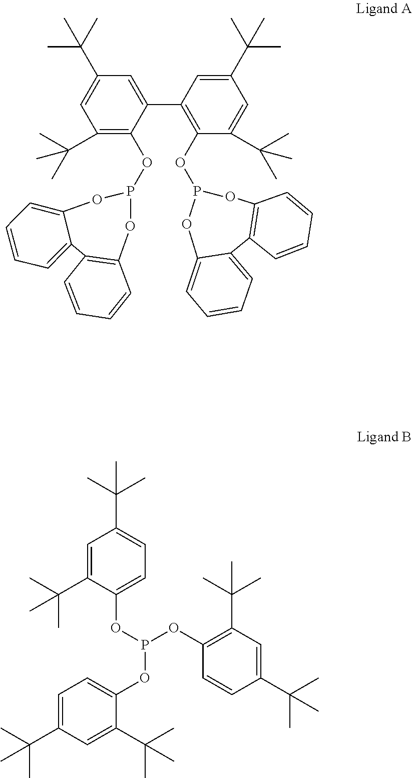 [SMILES: CC(C)(C)c1cc(-c2cc(C(C)(C)C)cc(C(C)(C)C)c2Op2oc3ccccc3c3ccccc3o2)c(Op2oc3ccccc3c3ccccc3o2)c(C(C)(C)C)c1 ; CC(C)(C)c1ccc(OP(Oc2ccc(C(C)(C)C)cc2C(C)(C)C)Oc2ccc(C(C)(C)C)cc2C(C)(C)C)c(C(C)(C)C)c1]

General fluid composition including heavies concentration of the OSN feed (the feed to the OSN separation membrane) and OSN permeate (the permeate from the OSN separation membrane) is determined by gas chromatography (GC) using techniques known to those having ordinary skill in the art.

### Component Quantitation is Based on External Standard Calibration.

The concentration of Ligands A or B in the OSN feed and OSN permeate samples is measured using high pressure liquid chromatography (HPLC) using techniques known to those having ordinary skill in the art.

### Quantitation is Based on External Standard Calibration.

Rhodium concentration in OSN feed and OSN permeate samples is determined via analysis using a Perkin Elmer Analyst 900 Atomic Absorption Analyzer. The samples (0.1-0.2 g) are diluted in 5 g of 99% 2-methoxyethanol (“methyl cellosolve”, Fisher) containing 0.2% lanthanum nitrate (Aldrich). Calibration standards are prepared in the same matrix.

### Hydroformylation Unit Description

Continuous hydroformylation is conducted in a liquid recycle reactor system comprised of three 1 liter stainless steel stirred tank reactors connected in series. Each reactor is equipped with a vertically mounted agitator and a circular tubular sparger located near the bottom of the reactor. Each sparger contains a plurality of holes of sufficient size to provide the desired gas flow into the liquid body in the reactor. The spargers are used for feeding the olefin and/or syngas to the reactor, and can also be used to recycle unreacted gases to each reactor. Each reactor has a silicone oil shell as a means of controlling reactor temperature. Reactors 1 to 2 and reactors 2 to 3 are further connected via lines to transfer any unreacted gases and lines to allow a portion of the liquid solution containing aldehyde product and catalyst to be pumped from reactor 1 to reactor 2 and from reactor 2 to reactor 3. Hence, the unreacted olefin of reactor 1 is further hydroformylated in reactor 2 and subsequently reactor 3. Each reactor also contains a pneumatic liquid level controller for maintaining the desired liquid level. Reactor 3 has a blow-off vent for removal of unreacted gases.

A portion of the liquid reaction fluid is continuously pumped from reactor 3 to a vaporizer, which comprises a heated vessel at reduced pressure. The effluent stream from the vaporizer is sent to a gas-liquid separator located at the bottom of the vaporizer, where vaporized aldehyde is separated from the non-volatile components of the liquid reaction solution. In this embodiment, the vaporizer and the gas-liquid separator comprise a product-catalyst separation zone. The vaporized aldehyde product is condensed and collected in a product receiver. A pneumatic liquid level controller controls the desired non-volatile component level, including catalyst to be recycled, at the bottom of the separator. The non-volatile effluent from the separator (tails stream) is pumped through a recycle line into Reactor 1. Gas compositions (mole %) are measured by gas chromatography (GC) and partial pressures are then calculated based on the total pressure using Raoult's law.

### General Procedure

The experiments with OSN separation membranes are conducted in a small lab-scale apparatus. A schematic of the lab scale apparatus is shown in FIG. 2.

The membrane cell 17 (OSN stage) contains a circular piece of flat sheet OSN separation membrane with a surface area of 12.97 cm² supported by a thin disc comprised of sintered stainless steel. The OSN feed is introduced to the membrane cell at 5 mL/min by a small piston pump 18 via line 19. The pressure within the cell is established by a back-pressure regulator 20 downstream of the membrane cell and monitored via pressure gauge 21. The OSN permeate is collected in a small bottle 22 and sampled for analysis 25c. The rate of permeate collection (permeate flux) is determined gravimetrically; calculations are then applied to allow the value to be expressed as liters of permeate generated per square meter of membrane per hour (L/m²/hr). The OSN retentate is recycled to the OSN feed tank 23 via line 24 unless otherwise noted. Isolation valves 25a-d are generally left on except for maintenance or sampling.

### Example 1

The hydroformylation unit described above is used to produce mixed C9 aldehydes from a feed of mixed octenes comprising 1-octene and linear internal isomers; the catalyst is comprised of rhodium and Ligand A. After several weeks of continuous operation the catalyst fluid contains an appreciable concentration of heavies. The fluid is drained from the hydroformylation unit, added to the OSN feed tank, and subjected to nanofiltration following the general procedure shown in FIG. 2 employing the o-NF2 OSN separation membrane (from Borsig Membrane Technology). The results are summarized in Tables 1A and 1B.

### Example 2

The procedure of Example 1 is repeated with the exception of the use of the o-NF1 OSN separation membrane (Borsig Membrane Technology) instead of the o-NF2 OSN separation membrane. The results are summarized in Tables 1A and 1B.

### Examples 3-4

A continuous hydroformylation run is conducted using a mixed octenes feed comprising 1-octene, linear internal olefins and branched internal olefins using the hydroformylation unit described above. The catalyst is comprised of rhodium and Ligand B. After several weeks of continuous operation, the catalyst fluid contains an appreciable concentration of heavies. The fluid is drained from the hydroformylation unit, added to the OSN feed tank, and subjected to nanofiltration using the general procedure shown in FIG. 2 employing the o-NF2 membrane. The results are summarized in Tables 1A and 1B.

TABLE 1A

| | |
|---|---|
| cell | Feed composition |

| | | pressure | Rhodium | Ligand | heavies |
|---|---|---|---|---|---|
| Ex. | membrane | (psig) | (ppm) | (wt. %) | (wt. %) |
| 1 | o-NF2 | 625 | 447.0 | 0.334 | 26.9 |
| 2 | o-NF1 | 250 | 502.0 | 1.138 | 28.1 |
| 3 | o-NF2 | 250 | 290.5 | 1.635 | 17.8 |
| 4 | o-NF2 | 100 | 322.5 | 1.762 | 18.5 |

TABLE 1B

| | permeate | Permeate composition | | | Rejection (%) | | |
|---|---|---|---|---|---|---|---|
| | flux | Rhodium | Ligand | heavies | | li- | |
| | (L/m²/hr) | (ppm) | (wt. %) | (wt. %) | rhodium | gand | heavies |
| Ex. | 5.84 | 78.2 | 0.124 | 19.5 | 82.5 | 66.0 | 25.5 |
| 1 | 3.34 | 104.2 | 0.769 | 25.9 | 79.3 | 32.5 | 8.0 |
| 2 | 2.74 | 59.8 | 0.378 | 14.8 | 79.4 | 41.8 | 17.1 |
| 3 | 2.21 | 45.4 | 1.068 | 17.4 | 86.0 | 39.0 | 6.0 |
| 4 | | | | | | | |

The results of Tables 1A and 1B show that the OSN feed may be provided to an OSN separation membrane to produce an OSN permeate stream comprising heavies and a lower rhodium concentration than the OSN feed. The organophosphorous ligand concentration in the OSN permeate stream is also reduced relative to the OSN feed. The Rejection (%) reported in Table 1B is contemplated to describe the tendency for the OSN separation membrane to retain the transition metal, organophosphorous ligand, or heavies within the OSN feed. Ideally, the Rejection will be high for rhodium and organophosphorous ligand, and low for heavies. The Rejection is calculated and expressed as a percentage. For example, if an OSN feed comprising rhodium at a concentration of 100 ppm is provided to an OSN stage and produces a permeate stream comprising 25 ppm rhodium, the rhodium rejection is calculated to be 75%.

It should be understood that the lab scale apparatus shown in FIG. 2 is designed to facilitate small scale experiments in a laboratory setting. Commercial implementations would vary as described elsewhere herein. For example, rather than using an OSN feed tank, a tails stream from a product-catalyst separation zone can be continuously provided to an OSN separation membrane, multiple OSN separation membranes can be used, etc.

What is claimed is:

1. A process for recovering rhodium from a hydroformylation process that comprises producing at least one aldehyde in a reaction zone, the reaction zone comprising a C₆ to C₂₂ olefin, hydrogen and carbon monoxide in the presence of a catalyst, wherein the catalyst comprises rhodium and an organophosphorus ligand, the process comprising:
(a) receiving a tails stream from a product-catalyst separation zone, wherein the tails stream comprises aldehydes, heavies, rhodium, and an organophosphorous ligand;
(b) providing at least a portion of the tails stream to at least one organic solvent nanofiltration (OSN) separation membrane, wherein a final permeate stream exits a final OSN separation membrane, the final permeate stream comprising aldehydes, heavies, rhodium, and an organophosphorous ligand, wherein the rhodium concentration in the final permeate stream is lower than the rhodium concentration in the tails stream; and
(c) incinerating the final permeate stream on-site to create a rhodium-containing ash.

2. The process of claim 1, wherein the process uses one OSN separation membrane.

3. The process of claim 1, wherein the process uses two OSN separation membranes.

4. The process of claim 3, wherein all of the permeate stream from the first OSN separation membrane is provided to the second OSN separation membrane.

5. The process of claim 1, wherein the incinerator in which the final permeate stream is incinerated to create the rhodium-containing ash is located within a ten mile radius of the product-catalyst separation zone.

6. The process of claim 1, further comprising recovering rhodium from the rhodium-containing ash.

7. The process of claim 1, further comprising passing at least a portion of retentate from at least one OSN separation membrane through a first OSN separation membrane.

8. The process of claim 1, wherein the final permeate stream is incinerated within 90 days of the tails stream leaving the product-catalyst separation zone.

9. The process of claim 1 further comprising treating the final permeate stream to be incinerated prior to incineration to recover residual aldehyde product.
